(19) United States
(12) Patent Application Publication
(10) Pub. No.: US 2014/0121645 A1
(43) Pub. Date: May 1, 2014

(54) METHODS AND APPARATUS FOR THE CVCS

(71) Applicant: Gordon Wayne Dyer, Hobbs, NM (US)

(72) Inventor: Gordon Wayne Dyer, Hobbs, NM (US)

(21) Appl. No.: 13/261,795

(22) PCT Filed: Jul. 16, 2012
(86) PCT No.: PCT/US2012/000324

§ 371 Date: Jan. 9, 2014

Related U.S. Application Data
(63) Continuation-in-part of application No. 13/199,012, filed on Aug. 16, 2011.

(51) Int. Cl. A61M 31/00; A61M 37/00

(52) U.S. Cl. CPC A61M 31/00; A61M 37/00
USPC 604/514; 604/278

(57) ABSTRACT

The present invention provides an indirect method and accompanying apparatus for supplying a high concentration of medicaments, particularly antibiotics, to the nasal sinuses by first loading the medicament into the cerebrospinal venous system (CVCS) via a Valsalva maneuver. Because the CVCS is a valveless, three-dimensional closed system, traditional physiological dogma such as veins always draining tissues does not always apply. Instead, because in its closed-system blood can flow in any direction, the blood of the CVCS and any medicaments that it contains will be drawn to any portion of it where there is increased outflow, such as the copious venousderived sinus fluid drainage present during nasal allergy or nasal infection. Thus, the very nasal congestion that impedes the effectiveness of direct medicament application, such as seen with nasal inhalers or systemic antibiotics, aids in applying the medicament indirectly to the nasal sinuses via the CVCS. Additionally, the present method has the benefit of delivering medicaments that, unlike present treatment regimens, are not limited solely to those medicaments that can be successfully absorbed from the G.I. tract. This means that, in the case of antibiotics, the bacteria infecting this portion of the CVCS will not be as resistant to treatment if they have not had prior exposure to this new line of antibiotics. Finally, if the infection extends to the eardrums, making the Valsalva maneuver painful, or if the patient is simply unusually sensitive, then earplugs to reduce the stress on the eardrums may be worn while the patient performs the Valsalva maneuver.

### CROSS-REFERENCES TO RELATED APPLICATIONS

This application is a continuation-in-part of, and claims the benefit of priority from, U.S. application Ser. No. 20020098154, filed Jul. 25, 2002, the entire content of which is incorporated herein by reference.

### BACKGROUND OF THE INVENTION

1. Field of the Invention

The present invention relates to applying medicaments to the cerebrospinal venous system. The present invention includes an applicator, medicaments that can be either/both water and fat-soluble and the use of the Valsalva maneuver for deposition of medicaments to the Eustachian tube for subsequent absorption into the cerebrospinal venous system. More particularly, the present invention relates to applying medicaments to the portions of a mammal's body that the cerebrospinal venous system venously supplies such as the nasal sinuses, eyes, teeth, brain, and mammalian column. Particular utility for the present invention is found in the area of facilitating delivery of medications (e.g., bacterial vaccines, sinusitis vaccines, antihistaminic agents, vasoconstricting agents, anti-bacterial agents, di-sodium cromolyn, etc.) to a difficult to reach area of the body, although other utilities are contemplated including other medicaments.

2. Description of Related Art

Inhalation devices are well known in the art for the dispensing of various kinds of medicament for inhalation by the patient. Inhalation devices come in a variety of different types such as metered dose inhalers (MDI), dry powder inhalers, vibrational inhalers, and nebulizers and are routinely used for the delivery of medicament for the treatment of respiratory disorders such as asthma and chronic inflammatory pulmonary disease.

A disadvantage of all such inhalers is that they place their topical, aerosolized medicaments in areas of the body which are not optimal for the treatment of dental, ocular, nasal sinus, brain, and spinal diseases.

For many years, it has been thought the venous return of blood from the head was carried out almost solely by the internal and external jugular veins. However, it is now known that in an upright position the jugular veins are collapsed and the majority of bloodflow from the head flows through a sponge-like collection of valveless veins most commonly referred to as the cerebrospinal venous system (CVCS) (Fasel J. The Craniocervical Venous System in Relation to Cerebral Venous Drainage. Am J Neuoradiol 23: 1500-1508, October 2002; Zamboni P. Doppler Haemodynmics of Cerebral Venous Return. Current Neurovascular Research, 2008, 5, 260-265). This large three-dimensional venous plexus system, also known as the mammalian venous plexus, is characterized by numerous freely-flowing bi-directional blood anastomoses interconnecting one portion of this overall plexus system to another. It extends from the brain to various blood plexuses and sinuses at the base of the brain, including the pterygoid plexus, and finally to intercommunicating internal and external mammalian venous plexuses that run along the entire length of the spine. However, the cerebrospinal venous system also includes the facial veins, superior and inferior ophthalmic veins, superior and inferior orbital veins, as well as the venous plexus of the maxillary sinus and thus freely communicates with all the paranasal sinuses as well as the orbit. It is theorized that this unique, sponge-like, valveless, ebbing and flowing blood plexus system's purpose is to insure that the brain maintains a steady temperature as well as a constant supply of blood regardless of head position, abdominal pressure or blood pressure. (Vega C. The Cerebrospinal Venous System: Anatomy, Physiology, and Clinical Implications. Medscape General Medicine. 2006; (18):53).

Because of its valvelessness, there is free communication between all elements of the CVCS and this barrierless communication explains hitherto unexplainable patterns of metastasis, infection and embolization where the disease agent travels “uphill” and from “far away” when thought from the point of view of traditional venous drainage (Prescher A. Infection transfer between the maxillary sinus and endocranium. Universitats-HNO-Klinik Essen, Universitat Duisburg-Essen; Vega C. The Cerebrospinal Venous System: Anatomy, Physiology, and Clinical Implications. Medscape General Medicine. 2006; (18):53; Amedee R. G. Orbital complications of sinusitis. J La State Med. Soc. 1997 April; 149(4):105-8). However, particle distribution throughout the CVCS is not governed solely by Browning motion. Focal changes of pressure, inflammation, or fluid drainage in one part of the CVCS influence the flow of blood in other adjacent parts of the CVCS and can induce focal phlebographic changes that, in the nose, result in rhinorrhea and nasal congestion (Kim, M. Cluster-like Headache Secondary to Cerebral Venous Thrombosis. Journal of Clinical Neurology. 2006 March; Vol. 2: 70-73; Karemaker, J. M. Human cerebral venous outflow pathway depends on posture and central venous pressure J Physiol 560.1 2004:317-327).

Normally, the internal part of the nose is venously drained by orbital, pterygoid and cavernous sinus portions of the CVCS. However, being valveless and having unique erectile-like venous sinusoids connected to these nasal venuoles, when this nasal venous complex is inflamed, such as during the common cold, allergic rhinitis, or rhinosinusitis, the resultant copious rhinorrhic fluid derived from this nasal venous complex means that there is likely venous flow reversal to help the nose shed itself of the offending viral particles and/or pollen antigens. Further, the concomitant rhinorrhic nasal congestion greatly reduces or completely eliminates any airflow through the nasal passages (Fairbanks D N F, Kaliner M. Nonallergic rhinitis and infection. In: Cummings C W, Fredrickson J M, Harker A L, Krause C J, Richardson M A, Schuller D E, eds. Otolaryngology Head and Neck Surgery, vol 2, ed 3. St. Louis: Mosby, 1998: 910-920; Baraniuk, J. Patholphysiology of nasal congestion. International Journal of General Medicine 2010:3 47-57). Therefore, because of this exudative fluid flow reversal and nasal congestion, any medicine simply sniffed into the nose would not be well absorbed but instead be quickly flushed out of the nose. Further, due to the nasal congestive obstruction, any such sniffed medicine would likely not be able to penetrate to the deeper areas of the nasal sinuses to begin with.

However, any medicine placed not in the nasal passages, but into the Eustachian tube via the use of a Valsalva maneuver would allow the medicine to have valveless free access to the CVCS because the Eustachian tube is surrounded and venously drained by the blood sponge that is the pterygoid plexus, a central portion of the CVCS with many interconnections to other parts of the CVCS (Bluestone, C. Eustachian tube: structure, function, role in otitis media, Volume 2 PMPH-USA, 2005: 45). Once absorbed into the CVCS the medicine could be used to treat a variety of dental, ocular, nasal, brain, and spinal diseases and disorders and, in addition, would have the benefit of crossing the blood/brain barrier that complicates medicating the brain and spinal chord. Given its unique vascular advantage via absorption into the CVCS, any topically applied medicament is inherently able to help in the treatment of a disease or medical disorder in an adjacent part of the body because all topical medicaments are eventually absorbed and thus distributed, at least to some degree, to adjacent parts of the mammal's body (Mealey, K. DVM, PhD Systemic Absorption of Topically Administered Drugs Scribd Inc.; Vol. 22, No. 7 Jul. 2000).

Because nasal inhalation of antibiotics has proven ineffective, the standard medical treatment for sinus infections currently is systemic antibiotics, coupled with concomitant use of systemic nasal decongestants. In more severe sinus infections, particularly if there is an accompanying allergic condition, the systemic antibiotic and decongestant therapy may be augmented with inhaled steroid or decongestant medicaments. Topical inhalation antibiotic therapy regimens have been proposed in the past, but without any apparent practical utility. However, some recent studies have again been studying the use of topical inhalation antibiotic therapy regimens. One of these was given FDA approval in October, 2000.

Sinus allergies are a major medical problem in the United States. Millions of dollars are spent every year on prescription and over-the-counter sinus allergy and sinus congestion/sinus pain medicaments. Because the allergy's inherent sinus congestion leads to a warm, moist environment with poor drainage, sinus allergies often lead to sinus infections. A shortcoming of the present standard oral regimen for sinus allergies is that chronic use of decongestants, antihistamines, and analgesics can, respectively, cause drowsiness, liver and/or kidney damage, and an increase in blood pressure. All of these shortcomings also apply to the present standard oral regimen for treating sinus infections. In addition, due to the recurrent nature of sinus infections and the high antibiotic dosages necessary to treat them, oral regimens for treating sinus infections lead to antibiotic-resistant bacteria.

Oral antibiotic therapies inherently induce antibiotic-resistant bacteria because the antibiotic is introduced not just to the bacteria that are causing the sinus infection, but to all the other endemic bacteria normally present in the body too, such as E. coli and Staph. aureus. This often-repeated-yet-unintended bacterial antibiotic exposure eventually leads to highly antibiotic-resistant bacteria that in turn cause future infections that are difficult to treat. Aggravating this difficulty, the inherent congestion of sinus infections impedes the delivery of the blood borne systemic antibiotic because the congestion impairs the flow of blood to the infected area. Trying to decrease the sinus congestion with steroid sprays, in order to increase the penetration of the systemic antibiotic, is often unsuccessful because the steroid concomitantly decreases the body's infection fighting ability. Thus the sinus infection worsens in spite of high amounts of powerful systemic antibiotics and often the only recourse is repeated sinus surgery.

In view of the foregoing, it would be desirable to place topical medicaments in an area of the body better situated for the treatment of dental, ocular, nasal sinus, brain, and spinal diseases than the nasal inhalation/G.I. tract absorption regimens currently being used. An advantage of the current invention is that it provides an alternative route of delivering a high concentration of medicaments to a large portion of the body. Another advantage of the current invention is that it provides an alternative group of medicaments for the treatment of dental, ocular, nasal sinus, and brain diseases or disorders than are currently being used such as those medicaments that are not well absorbed through the G.I. tract or capable of passing across the blood/brain barrier. Another advantage of the current invention is that, while basically being a reconfigured inhaler that is set to be triggered on exhalation rather than inhalation and thus enabled by all existing nasal inhaler technology known to the art, it provides an alternative, or supplementary, means of treating dental, ocular, nasal sinus, and brain diseases or disorders to the standard oral treatment route currently being used by physicians.

The scope of the present invention includes all devices for delivery and actuation of aerosolized medicaments known to the art including, but not limited to, U.S. Pat. Nos. 5,694,920, 6,026,809, 6,142,146, all by Abrams and Gumaste, U.S. Pat. No. 3,948,264 by Wilke et al., U.S. Pat. No. 6,971,383 by Hickey et al., U.S. Pat. No. 7,117,867 by Cox et al., U.S. Pat. No. 6,901,929 by Burr et al., U.S. Pat. No. 6,779,520 by Genova et al., U.S. Pat. No. 6,748,944 by DellaVecchia et al., U.S. Pat. No. 5,590,645 by Davies et al., and U.S. Pat. No. 7,963,154 by Obermeier, et al. The above patents provide an overview of various aerosolization devices and timing techniques but differ from the present invention because they are used for inhalation rather than exhalation. Further background information on aerosolized medicaments including nebulizers, metered-dose inhalers (MDI), and dry powder inhalation devices included within the scope of the present invention can be found in Wolff et al., Generation of Aerosolized Drugs, J. Aerosol: Med. pp. 89-106 (1994); Prime et al., Review of Dry Powder Inhalers, 26 Adv. Drug Delivery Rev., pp. 51-58 (1997); and Hickey et al., A new millennium for inhaler technology, 21 Pharm. Tech., n. 6, pp. 116-125 (1997).

A metered-dose inhaler (MDI) means a device that delivers a specific amount of medication in the form of a short burst of aerosolized medicine that is inhaled by the patient.

A nebulizer means a device that uses oxygen, compressed air or ultrasonic power to break up medical solutions/suspensions into small aerosol droplets generally having diameters of 1-5 micrometers, which are inhaled by the patient.

A Valsalva maneuver means to forcefully exhale air from the lungs while keeping the mouth and nose closed in order to force open the Eustachian tube by means of pressurized lung air. Alternatively, this exhalation of air may be mechanically supplied while keeping the mouth and nose closed in order to force open the Eustachian tube.

A pressure sensor means a device that measures the pressure of gases or liquids and generates an electrical signal as a function of the pressure imposed. When pressure is applied to the pressure sensor, the sensor acts to complete or break an electrical circuit. Examples of suitable pressure sensors include: piezoresistive strain gauges using silicon (monocrystalline), polysilicon thin film, bonded metal foil, thick film, and sputtered thin film; capacitive pressure sensors that using a diaphragm and pressure cavity to create a variable capacitor to detect strain due to applied pressure; electromagnetic pressure sensors that measure the displacement of a diaphragm by means of changes in inductance (reluctance), LVDT, Hall Effect, or by eddy current principle; piezoelectric sensors that uses the piezoelectric effect to measure pressure, acceleration, strain or force by converting them to an electrical charge; optical sensors that use of the physical change of an optical fiber to detect strain due to applied pressure, for example a fiber bragg grating; resonant sensors that uses the changes in resonant frequency in a sensing mechanism to measure stress, or changes in gas density, caused by applied pressure to, for example, vibrating wire, vibrating cylinders, quartz, and silicon MEMS; thermal pressure sensors that use the changes in thermal conductivity of a gas due to density changes to measure pressure for example a Pirani gauge; and, ionization pressure sensors that measure the flow of charged gas particles (ions) which varies due to density changes to measure pressure, for hot and cold cathode gauges.

A mammal means any air-breathing animal characterized by the possession of a mouth, nostrils, CVS, and a Eustachian tube.

A liposome means an artificially prepared vesicle made of a lipid bilayer which can be filled with medicaments for the delivery medicaments for the treatment of mammalian diseases and disorders.

A microsphere means a small spherical particle whose diameter ranges from about 1 to 1000 μm that can be made out of polystyrene.

A tilt sensor means a device made up of a cavity and an electrically conductive mass inside the cavity, such as a blob of mercury or rolling ball which can freely move by force of gravity from one end of the cavity to the other. One end of the cavity has two conductive elements (poles) such that, when the tilt sensor is oriented so that its conductive end is downwards, the force of gravity pulls the conductive mass onto the poles and shorts them, thereby acting as a switch throw.

The foregoing description is intended to be illustrative and is not to be taken as limiting. Other variations within the spirit and scope of this invention are possible and will be apparent to those skilled in the art.

### SUMMARY OF THE INVENTION

In one embodiment, the present invention provides a method for using a device in conjunction with or after a mammal's Valsalva maneuver exhalation. The exhaler has a body and a nozzle used for applying a medicament to the Eustachian tube of a mammal having nostrils for subsequent venous absorption into the mammal's cerebrospinal venous system (CVCS). The exhaler uses pressure/propellant force to transmit medicament from a medicament reservoir through the exhaler's nozzle and into the mammal's Eustachian tube opening. The method comprises placing the nozzle of the exhaler adjacent to the opening of the Eustachian tube, and then using the pressure force of the exhaler to transmit the medicament from the reservoir and through the nozzle to the opening of the Eustachian tube of the mammal. And then performing a Valsalva maneuver, either in conjunction with or after the Valsalva maneuver, to exhalingly place the medicament into the Eustachian tube for subsequent venous absorption into the CVCS. Medicaments can also be delivered in combination with other medicaments.

The present invention includes, but is not limited to, all the medicament delivery technology taught by U.S. Pat. Nos. 5,694,920, 6,026,809, 6,142,146, all by Abrams and Gumaste, U.S. Pat. No. 3,948,264 by Wilke et al., U.S. Pat. No. 6,971,383 by Hickey et al., U.S. Pat. No. 7,117,867 by Cox et al., U.S. Pat. No. 6,901,929 by Burr et al., U.S. Pat. No. 6,779,520 by Genova et al., U.S. Pat. No. 6,748,944 by DellaVecchia et al., U.S. Pat. No. 5,590,645 by Davies et al., and U.S. Pat. No. 7,963,154 by Obermeier, et al.

The present invention's appropriate medicaments include, but are not limited to analgesics, e.g., codeine, dihydromorphine, ergotamine, fentanyl or morphine; antiinfectives e.g., cephalosporins, fluoroquinolones, penicillins, streptomycin, sulphonamides, tetracyclines and pentamidine; antihistamines, e.g., methapyrilene; anti-inflammatories, e.g., ketoraolac tromethamine, nepafenac, diclofenac, bromfenac, beclomethasone dipropionate, fluticasone propionate, flunisolide, budesonide, rofleponide, mometasone furoate or triamcinolone acetonide; anticholinergics, e.g., ipratropium, tiotropium, atropine or oxitropium; hormones, e.g., cortisone, hydrocortisone or prednisolone; anti-glaucoma e.g. carbonic anhydrase inhibitors and beta-blockers; anti-seizure medications; therapeutic proteins and peptides, e.g., insulin or glucagon; and various neurological agents such as gabapentin, an anticonvulsant memantine, levetiracetam, 3,4-diaminopyridine, 4-aminopyridine, baclofen, meclozine and carbonic anhydrase inhibitors. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

In one embodiment, the method further comprises removing the exhaler from the mammal before performing the Valsalva maneuver.

In a preferred embodiment, the method further comprises placing the exhaler in the mammal's nostrils, which the exhaler's body is adapted to receive and block any exhalation through, the mammal's nostrils and the exhaler remains in the mammal's nostrils during the Valsalva maneuver.

In another embodiment, the method further comprises placing the exhaler in the mammal's nostrils, which the exhaler's body is adapted to receive and block any exhalation or inhalation through, the mammal's nostrils and the exhaler remains in the mammal's nostrils during the Valsalva maneuver.

In another embodiment, the method further comprises placing the exhaler in the mammal's mouth instead of nostrils. The exhaler's body is adapted to receive, and block any exhalation through, the mammal's mouth, and the exhaler remains in the mammal's mouth during the Valsalva maneuver.

In another embodiment, the method further comprises the medicament being a suspension medium composed of a pharmaceutically acceptable propellant, one or more biologically active substances, one or more active agent particles, and one or more suspending particles. In this embodiment the active agent particles aid in the distribution of the biologically active substance in the vertebrate and also associate with the suspending particles to co-suspend the biologically active substance. The medicaments of the present inventions includes the use of co-suspensions of active agent particles and suspending particles to provide chemical stability, suspension stability and enhance the delivery of the active agent to the mammal. Patent references teaching suitable methods for obtaining the included active agent particles and suspending particles are described, for example, in U.S. Pat. No. 6,063,138, U.S. Pat. No. 5,858,410, U.S. Pat. No. 5,851,453, U.S. Pat. No. 5,833,891, U.S. Pat. No. 5,707,634, and International Patent Publication No. WO 2007/009164.

Examples of suspending particles encompassed by the present invention include, but are not limited to: monosaccharides such as fructose, galactose, glucose, D-mannose, sorbose; disaccharides, such as sucrose, lactose, trehalose, cellobiose; cyclodextrins, such as 2-hydroxypropyl-.beta.-cyclodextrin; polysaccharides, such as raffinose, maltodextrins, dextrans, starches, chitin, chitosan, inulin; and saturated and unsaturated lipids, nonionic detergents, nonionic block copolymers, and ionic surfactants.

Patent references teaching the present invention's pharmaceutically acceptable propellants include, but are not limited to, GB 9002351, U.S. Pat. No. 5,182,097, EP 372777, DE 4003272A1, DE 3905726A1, DE 3905726A1 U.S. Pat. No. 5,891,419, U.S. Pat. No. 5,439,670, U.S. Pat. No. 5,474,759, U.S. Pat. No. 5,492,688, and air, carbon dioxide, and nitrogen.

In another embodiment, the method further comprises the medicament being composed of a pharmaceutically acceptable propellant, one or more biologically active substances, and a preparation containing liposomes or microspheres. In this embodiment the biologically active substance is first contacted with the liposomes or microspheres in an aqueous medium before being propelled by the propellant. Examples of propellants encompassed by the current invention include, but are not limited to, hydrofluoroalkanes (HFAs), perfluorinated compounds (PFCs), and chlorofluorocarbons (CFCs). Patent references teaching suitable methods for obtaining the liposomes and microspheres included in the present invention are described, for example, in U.S. Pat. No. 5,595,756, U.S. Pat. No. 6,613,352, U.S. Pat. No. 6,815,432, U.S. Pat. No. 5,976,567, U.S. Pat. No. 7,169,410, U.S. Pat. No. 4,744,989, U.S. Pat. No. 4,224,179, U.S. Pat. No. 5,599,889, U.S. Pat. No. 5,260,002, U.S. Pat. No. 5,643,506, U.S. Pat. No. 7,951,402, U.S. Pat. No. 7,727,555, and U.S. Pat. No. 7,462,366.

In accordance with a preferred embodiment, the present invention includes an exhaler, for use in conjunction with a Valsalva maneuver to open the mammal's Eustachian tube. The exhaler is used for applying a medicament to the cerebrospinal venous system (CVCS) of a mammal. The exhaler is capable of exerting pressure force and comprises: a body adapted to receive, and block any exhalation through, the mammal's nostrils, a medicament reservoir coupled to this pressure force, and a nozzle adapted to receive, and transmit medicaments to, the mammal's Eustachian tube opening. When the Valsalva maneuver is performed to open the Eustachian tube, and with the body of the exhaler blocking the mammal's nostrils and the nozzle of the exhaler adjacent to the now-opened Eustachian, the pressure force of the exhaler transfers the medicament from the reservoir and through the nozzle to the now open Eustachian tube for absorption into the CVCS, which venously drains the Eustachian tube. Medicaments can also be delivered in combination with other medicaments.

In another embodiment, the exhaler further comprises having both a meter, that fluidly communicates selectively between the reservoir and the mammal, for metering an amount of medicament available to the pressure force of the exhaler and a electromechanical actuating means coupled to an exhalation sensor, which triggers, activates, and controls the electromechanical actuating means for sensing the exhalation of the mammal. The electromechanical actuating means of the present invention can be, but is not limited to, a spring and/or a lever, a solenoid, a wire, a strip, a coil, or a tube and can include the electromechanical actuating means being composed of an alloy which is reversibly deformable in response to heat or an alloy which is reversibly deformable in response to a magnetic field. Suitable magnetic shape memory alloys included in the present invention are described in, but not limited to, U.S. Pat. No. 5,958,154, U.S. Pat. No. 6,157,101, and U.S. Pat. No. 6,515,382. In another aspect, suitable heat memory alloys encompassed in the present invention's electromechanical actuating means include multiple layers of different metals (e.g. bimetallic strips), each material having a different coefficient of thermal expansion, piezoelectric materials including piezoelectric ceramics (e.g. compounds of lead zirconate and lead titanate), piezoelectric crystals such as polycrystalline ferroelectric materials with the perovskite structure, a nickel-titanium alloy (Cu and Nb may be present in trace amounts), a copper-aluminium-nickel alloy, and a copper-zinc-aluminium alloy. Suitable heat shape memory alloys included in the present invention are described in U.S. Pat. No. 5,641,364, U.S. Pat. No. 5,865,418, U.S. Pat. No. 5,211,371, and U.S. Pat. No. 6,321,845.

The present invention also includes having the actuation of the pressure force used to transmit the metered amount of medicament from the reservoir to the mammal being responsive to the exhalation sensor. The electromechanical actuating means, in response to the exhalation sensor, actuates the meter at a predetermined trigger point in time relative to the mammal's Valsalva maneuver exhalation in order to achieve the maximum possible distribution of the medicament into the Eustachian tube. For example, in a preferred embodiment, the actuation is triggered by the sensor at the same moment in time as the Eustachian tube is opened in order to take advantage of the vacuum-like Venturi effect created when the Eustachian tube is opened and thus help suck the medicament into the tube for later absorption into the CVCS. The meter may comprise a valve (for example, a linear or rotary valve) and/or a piston and/or a load cell. The meter may also comprise a plunger, such as might exist in a syringe, or a diaphram. Embodiments including multiple plungers and multiple syringe chambers are also envisaged. The meter comprises at least one metering chamber. In one embodiment, upon actuation of the meter, the metering chamber moves into fluid communication with the reservoir. Patent references teaching suitable metering, coupling and actuating techniques included in the present invention are described in, but not limited to, U.S. Pat. No. 4,534,343, U.S. Pat. No. 4,852,561, U.S. Pat. No. 5,040,527, U.S. Pat. No. 5,263,475, U.S. Pat. No. 5,320,714, U.S. Pat. No. 5,341,801, U.S. Pat. No. 5,431,154, U.S. Pat. No. 5,447,150, U.S. Pat. No. 5,497,944, U.S. Pat. No. 3,981,197, U.S. Pat. No. 3,935,634, U.S. Pat. No. 3,995,247, U.S. Pat. No. 4,016,644, U.S. Pat. No. 4,023,562, U.S. Pat. No. 4,406,992, U.S. Pat. No. 5,518,951, U.S. Pat. No. 5,589,810, U.S. Pat. No. 5,867,886, U.S. Pat. No. 6,319,743, U.S. Pat. No. 3,935,636, U.S. Pat. No. 4,745,812, U.S. Pat. No. 4,745,812, U.S. Pat. No. 4,849,730, U.S. Pat. No. 5,505,093, U.S. Pat. No. 5,886,615, U.S. Pat. No. 4,685,469, U.S. Pat. No. 4,554,927, U.S. Pat. No. 5,973,590, U.S. Pat. No. 4,685,469, U.S. Pat. No. 4,967,600, U.S. Pat. No. 4,744,252, U.S. Pat. No. 4,227,418, U.S. Pat. No. 4,257,274, U.S. Pat. No. 4,287,553, U.S. Pat. No. 4,292,659, U.S. Pat. No. 4,322,977, U.S. Pat. No. 4,332,000, U.S. Pat. No. 4,336,567, U.S. Pat. No. 4,454,418, U.S. Pat. No. 6,191,414, U.S. Pat. No. 5,844,667, U.S. Pat. No. 5,877,426, U.S. Pat. No. 4,932,262, U.S. Pat. No. 4,040,290, U.S. Pat. No. 4,062,354, U.S. Pat. No. 4,072,927, U.S. Pat. No. 4,178,804, U.S. Pat. No. 4,149,422, U.S. Pat. No. 4,739,664, U.S. Pat. No. 4,297,872 U.S. Pat. No. 4,311,053, U.S. Pat. No. 4,435,986, U.S. Pat. No. 4,547,691, U.S. Pat. No. 4,409,586, U.S. Pat. No. 5,227,798, U.S. Pat. No. 6,823,718, U.S. Pat. No. 5,702,592, U.S. Pat. No. 4,995,264, U.S. Pat. No. 5,583,297, U.S. Pat. No. 5,633,465, U.S. Pat. No. 6,227,056, U.S. Pat. No. 5,617,845. U.S. Pat. No. 4,222,263, U.S. Pat. No. 5,183,056, U.S. Pat. No. 6,584,846, U.S. Pat. No. 4,660,018, U.S. Pat. No. 6,765,394, U.S. Pat. No. 5,596,272, U.S. Pat. No. 4,406,272, U.S. Pat. No. 4,508,092, U.S. Pat. No. 4,821,560, U.S. Pat. No. 3,946,615, U.S. Pat. No. 3,958,558 U.S. Pat. No. 4,112,777 U.S. Pat. No. 4,161,886, U.S. Pat. No. 4,412,454, U.S. Pat. No. 4,866,988, U.S. Pat. No. 5,450,853, U.S. Pat. No. 4,663,964, U.S. Pat. No. 4,484,173, U.S. Pat. No. 4,487,074, U.S. Pat. No. 4,340,877, U.S. Pat. No. 4,352,085, U.S. Pat. No. 4,936,148, U.S. Pat. No. 4,905,520, U.S. Pat. No. 3,995,493 and U.S. Pat. No. 4,513,609.

In one embodiment, the exhalation sensor comprises an exhalation-movable element which is movable in response to the exhalation of the mammal. Preferably, the exhalation-movable element consists of a vane, a sail, a piston, a diaphragm, a bourbon tube, a bellows, or an impeller. Movement of the exhalation-movable element may be detectable by any suitable technique for detecting movement known to the art. Suitable exhalation sensor techniques include optical detectors, magnetic detectors or detectors using detection of capacitative effects.

Optical detectors may be used to detect movement of the exhalation-movable element by providing the element with a patterned outer surface, for example strips in a barcode type arrangement, and locating the optical detector so that it points towards the patterned surface. Movement of the exhalation-movable element alters the amount of the light source which reflects back onto the optical detector as the beam passes over the patterned surface. The strips may be arranged so that the direction of movement of the element can be detected. Patent references teaching suitable methods for the optical detectors included in the present invention are described in, but not limited to, U.S. Pat. No. 7,463,796, U.S. Pat. No. 7,459,671, U.S. Pat. No. 7,161,586, U.S. Pat. No. 5,291,013, U.S. Pat. No. 5,276,322, U.S. Pat. No. 5,241,300, and U.S. Pat. No. 5,212,379.

The present invention's magnetic detectors/sensors may be used to detect the movement of exhalation-movable element by the use of a magnetic switch device. A reader is located on the dispenser and magnetic material embedded within the exhalation-movable element (or vice-versa). Movement of the exhalation-movable element results in a change of the magnetic field experienced by the reader. Alternatively, electromagnetic pressure sensors/detectors, whereby a semiconductor measures the strength of the magnetic field of the magnetic material on the exhalation-movable element by means of changes in inductance (reluctance), LVDT, Hall Effect, or by eddy current principle are also encompassed by the present invention. The present invention includes, but is not limited to, all the detector technology taught by U.S. Pat. No. 4,222,263, U.S. Pat. No. 5,183,056, U.S. Pat. No. 6,584,846, U.S. Pat. No. 4,660,018, U.S. Pat. No. 6,765,394, U.S. Pat. No. 5,596,272, U.S. Pat. No. 4,406,272, U.S. Pat. No. 4,508,092, U.S. Pat. No. 4,821,560, U.S. Pat. No. 3,946,615, U.S. Pat. No. 3,958,558 U.S. Pat. No. 4,112,777 U.S. Pat. No. 4,161,886, U.S. Pat. No. 4,412,454, U.S. Pat. No. 4,866,988, U.S. Pat. No. 5,450,853, U.S. Pat. No. 4,663,964, U.S. Pat. No. 4,484,173, U.S. Pat. No. 4,487,074, U.S. Pat. No. 4,340,877, U.S. Pat. No. 4,352,085, U.S. Pat. No. 4,936,148, U.S. Pat. No. 4,905,520, U.S. Pat. No. 3,995,493 and U.S. Pat. No. 4,513,609.

The present invention also includes the exhalation sensor being comprised of a pressure sensor for sensing the pressure profile associated with the exhalation of the mammal. Any pressure transducer known to the art is an example of such a suitable pressure sensor included in the present invention. Other examples of suitable pressure sensors include: piezoresistive strain gauges using silicon (monocrystalline), polysilicon thin film, bonded metal foil, thick film, and sputtered thin film; capacitive pressure sensors that using a diaphragm and pressure cavity to create a variable capacitor to detect strain due to applied pressure; piezoelectric sensors that uses the piezoelectric effect to measure pressure, acceleration, strain or force by converting them to an electrical charge; optical sensors that use of the physical change of an optical fiber to detect strain due to applied pressure, for example a fiber bragg grating; resonant sensors that uses the changes in resonant frequency in a sensing mechanism to measure stress, or changes in gas density, caused by applied pressure to, for example, vibrating wire, vibrating cylinders, quartz, and silicon MEMS; thermal pressure sensors that use the changes in thermal conductivity of a gas due to density changes to measure pressure for example a Pirani gauge; and, ionization pressure sensors that measure the flow of charged gas particles (ions) which varies due to density changes to measure pressure, for hot and cold cathode gauges.

The present invention includes, but is not limited to, all the pressure sensor technology taught by U.S. Pat. No. 3,981,197, U.S. Pat. No. 3,935,634, U.S. Pat. No. 3,995,247, U.S. Pat. No. 4,016,644, U.S. Pat. No. 4,023,562, U.S. Pat. No. 4,406,992, U.S. Pat. No. 5,518,951, U.S. Pat. No. 5,589,810, U.S. Pat. No. 5,867,886, U.S. Pat. No. 6,319,743, U.S. Pat. No. 3,935,636, U.S. Pat. No. 4,745,812, U.S. Pat. No. 4,745,812, U.S. Pat. No. 4,849,730, U.S. Pat. No. 5,505,093, U.S. Pat. No. 5,886,615, U.S. Pat. No. 4,685,469, U.S. Pat. No. 4,554,927, U.S. Pat. No. 5,973,590, U.S. Pat. No. 4,685,469, U.S. Pat. No. 4,967,600, U.S. Pat. No. 4,744,252, U.S. Pat. No. 4,227,418, U.S. Pat. No. 4,257,274, U.S. Pat. No. 4,287,553, U.S. Pat. No. 4,292,659, U.S. Pat. No. 4,322,977, U.S. Pat. No. 4,332,000, U.S. Pat. No. 4,336,567, U.S. Pat. No. 4,454,418, U.S. Pat. No. 6,191,414, U.S. Pat. No. 5,844,667, U.S. Pat. No. 5,877,426, U.S. Pat. No. 4,932,262, U.S. Pat. No. 4,040,290, U.S. Pat. No. 4,062,354, U.S. Pat. No. 4,072,927, U.S. Pat. No. 4,178,804, U.S. Pat. No. 4,149,422, U.S. Pat. No. 4,739,664, U.S. Pat. No. 4,297,872 U.S. Pat. No. 4,311,053, U.S. Pat. No. 4,435,986, U.S. Pat. No. 4,547,691, U.S. Pat. No. 4,409,586, U.S. Pat. No. 5,227,798, U.S. Pat. No. 6,823,718, U.S. Pat. No. 5,702,592, U.S. Pat. No. 4,995,264, U.S. Pat. No. 5,583,297, U.S. Pat. No. 5,633,465, and U.S. Pat. No. 6,227,056.

In another aspect, the sensor comprises an airflow sensor for sensing the airflow profile associated with the exhalation of a patient. Patent references teaching suitable methods for the present invention's airflow sensor include U.S. Pat. No. 7,744,542, U.S. Pat. No. 5,379,650, U.S. Pat. No. 6,543,449, U.S. Pat. No. 6,761,165, U.S. Pat. No. 7,000,612, and U.S. Pat. No. 7,343,823.

In another aspect, the sensor comprises a temperature sensor for sensing the temperature profile associated with the exhalation of a patient. Patent references teaching suitable methods for the present invention's temperature sensor include U.S. Pat. No. 7,744,542, U.S. Pat. No. 3,785,774, U.S. Pat. No. 4,036,211, U.S. Pat. No. 6,968,743, U.S. Pat. No. 5,022,766, and U.S. Pat. No. 7,347,826.

In another aspect, the sensor comprises a moisture sensor for sensing the moisture profile associated with the exhalation of a patient. Patent references teaching suitable methods for the present invention's temperature sensor include U.S. Pat. No. 4,438,480, U.S. Pat. No. 4,482,581, U.S. Pat. No. 4,532,016, U.S. Pat. No. 4,816,748, U.S. Pat. No. 5,227,636, and U.S. Pat. No. 4,990,781.

In another embodiment, the present invention further comprises the pressure force of the exhaler being supplied by the mammal.

In another embodiment, the present invention's exhaler further comprises the medicament being a suspension medium composed of a pharmaceutically acceptable propellant; one or more biologically active substances; one or more active agent particles; and one or more suspending particles, wherein the active agent particles and suspending particles associate together to co-suspend the biologically active substance. In this embodiment the active agent particles aid in the distribution of the biologically active substance in the mammal and also associate with the suspending particles to co-suspend the biologically active substance. The medicaments of the present invention includes the use of co-suspensions of active agent particles and suspending particles to provide chemical stability, suspension stability and enhance the delivery of the active agent to the mammal. Patent references teaching suitable methods for obtaining the active agent particles and suspending particles included in the present invention are described, for example, in U.S. Pat. No. 6,063,138, U.S. Pat. No. 5,858,410, U.S. Pat. No. 5,851,453, U.S. Pat. No. 5,833,891, U.S. Pat. No. 5,707,634, and International Patent Publication No. WO 2007/009164.

Examples of suspending particles encompassed by the present invention's exhaler include, but are not limited to: monosaccharides such as fructose, galactose, glucose, D-mannose, sorbose; disaccharides, such as sucrose, lactose, trehalose, cellobiose; cyclodextrins, such as 2-hydroxypropy.beta.-cyclodextrin; polysaccharides, such as raffinose, maltodextrins, dextrans, starches, chitin, chitosan, inulin; and saturated and unsaturated lipids, nonionic detergents, nonionic block copolymers, and ionic surfactants. Examples of propellants encompassed by the current invention include, but are not limited to, hydrofluoroalkanes (HFAs), perfluorinated compounds (PFCs), and chlorofluorocarbons (CFCs). Patent references teaching some of the present invention's pharmaceutically acceptable propellants include, but are not limited to, GB9002351, US5182097, EP372777, DE4003272A1, DE3905726A1, DE3905726A1 U.S. Pat. No. 5,891,419 U.S. Pat. No. 5,439,670 U.S. Pat. No. 5,474,759 U.S. Pat. No. 5,492,688 and also air, carbon dioxide, nitrogen, and inert gas.

In another embodiment, the present invention further comprises the medicament being composed of: a pharmaceutically acceptable propellant, one or more biologically active substances, and a preparation containing liposomes or microspheres. In this embodiment the biologically active substance is first contacted with the liposomes or microspheres in an aqueous medium before being propelled by the propellant. Patent references teaching suitable methods for obtaining the liposomes and microspheres included in the present invention are described, for example, in U.S. Pat. No. 5,595,756, U.S. Pat. No. 6,613,352, U.S. Pat. No. 6,815,432, U.S. Pat. No. 5,976,567, U.S. Pat. No. 7,169,410, U.S. Pat. No. 4,744,989, U.S. Pat. No. 4,224,179, U.S. Pat. No. 5,599,889, U.S. Pat. No. 5,260,002, U.S. Pat. No. 5,643,506, U.S. Pat. No. 7,951,402, U.S. Pat. No. 7,727,555, and U.S. Pat. No. 7,462,366.

The present invention also includes having the electromechanical actuating means being coupled to a tilt sensor so that the actuation of the pressure force used to transmit the metered amount of medicament from the reservoir to the mammal is limited by the tilt sensor to a inclination range of between substantially zero to substantially sixty degrees relative to the sagittal and coronal planes of the mammal. In a preferred embodiment, electromechanical actuating means is coupled to both a tilt sensor and a pressure sensor such that actuation of the pressure force used to transmit the metered amount of medicament from the reservoir to the mammal is possible only when the mammal's tilt and exhalation pressure are both optimal for maximum transmission of the exhaler's medicament to the mammal's Eustachian tube. In self-actuating embodiments of the present invention, a buzzer and/or bell may be used to tell the mammal when the tilt and pressure conditions are optimal for actuating the transmission of the medicament from the exhaler. Patent references teaching suitable methods for the present invention's tilt sensor are described in, but not limited to, U.S. Pat. No. 3,097,565, U.S. Pat. No. 2,303,360, U.S. Pat. No. 2,540,974, and U.S. Pat. No. 2,427,902.

Preferably, the exhalation sensor triggers/actuates/starts the electromechanical actuating means at a predetermined trigger point in time relative to the mammal's Valsalva maneuver. For example, the trigger point may be during the beginning middle stage, or end of the mammal's exhalation cycle.

The present invention includes having the medicament be both water-soluble and fat-soluble.

The present invention includes having the medicament be applied while the patient is wearing earplugs.

The present invention includes having the medicament be selected from the group consisting of chloramphenicol, ciprofloxacin, gentamicin, norfloxacin, ofloxacin, tobramycin, polymyxin B, neomycin, trimethoprim, natamycin, povidone-iodine, diclofenac, ketorolac, flurbiprofen, suprofen, idoxuridine, trifluridine, cidofovir, acyclovir, famciclovir, valacyclovir, cromolyn sodium, ketorolac tromethamine, levocabastine ketotifen, iodoxamide, emedastine, olopatadine, loteprednol etabonate, pemerolast potassium, levofloxacin, amphotericin B, nystatin, miconazole, and ketoconazole.

The present invention includes having the medicament be a spray of liquid.

The present invention includes having the medicament be a drop of liquid.

The present invention includes having the medicament be a powder.

The present invention includes having the medicament be an antifungal medicament.

The present invention includes having the medicament be a mast cell stabilizer.

The present invention includes having the medicament be a non-steroidal anti-inflammatory drug.

The present invention includes having the medicament be a corticosteroid.

The present invention includes having the medicament be an antibiotic.

The present invention also includes having a medicament applicator that uses a propellant gas selected from the group consisting of nitrogen gas, helium gas, inert gas, and air.

The present invention includes having the applicator use a medicament that is both water-soluble and fat-soluble.

The present invention includes having the applicator use a medicament that is an antifungal medicament.

The present invention includes having the applicator use a medicament that is an antibiotic.

The present invention includes having the applicator use a medicament that is a mast cell stabilizer.

The present invention includes having the applicator use a medicament that is a corticosteroid.

The present invention includes having the applicator be used while the patient wears at least one earplug in his ear canal.

The medicaments used by this invention's exhaler can also include, but are not limited to: chloramphenicol, ciprofloxacin, gentamicin, norfloxacin, ofloxacin, tobramycin, polymyxin B, neomycin, trimethoprim, natamycin, povidone-iodine, diclofenac, ketorolac, flurbiprofen, suprofen, idoxuridine, trifluridine, cidofovir, acyclovir, famciclovir, valacyclovir, cromolyn sodium, ketorolac tromethamine, levocabastine ketotifen, iodoxamide, emedastine, olopatadine, loteprednol etabonate, pemerolast potassium, levofloxacin, amphotericin B, nystatin, miconazole, and ketoconazole.

The present invention includes the use of any suitable diagnostic, prophylactic or therapeutic agent. The medicament may be a pure drug, but more commonly, it is a drug mixed with a bulking agent (excipient), for example, lactose.

Additional medicaments may be engineered with particular densities, size ranges, or characteristics. Particles may comprise active agents, surfactants, wall forming materials, or other components considered desirable by those of ordinary skill.

I claim:

1. In accordance with the present invention, a method using an exhaler having a body and a nozzle is provided for applying a medicament to the Eustachian tube of a mammal having nostrils for subsequent venous absorption into the mammal's cerebrospinal venous system (CVCS), whereby the exhaler is capable of exerting pressure force and has a medicament reservoir coupled to this pressure force, and the exhaler's nozzle is adapted to receive, and transmit medicaments to, the mammal's Eustachian tube opening, the method comprising: placing the nozzle of the exhaler adjacent to the opening of the Eustachian tube, using the pressure force of the exhaler to transfer the medicament from the reservoir and through the nozzle to the opening of the Eustachian tube of the mammal, performing a Valsalva maneuver to exhalingly drive the medicament into the Eustachian tube for subsequent venous absorption into the CVCS.

2. The method of claim 1, wherein the exhaler is removed from the mammal before performing the Valsalva maneuver.

3. The method of claim 1, wherein the exhaler is placed in the mammal's nostrils, the exhaler's body is adapted to receive, and block any exhalation through, the mammal's nostrils and the exhaler remains in the mammal's nostrils during the Valsalva maneuver.

4. The method of claim 1 wherein the exhaler is placed in the mammal's mouth, the exhaler's body is adapted to receive, and block any exhalation through, the mammal's mouth and the exhaler remains in the mammal's mouth during the Valsalva maneuver.

5. The method of claim 1, wherein the medicament is a suspension medium composed of a pharmaceutically acceptable propellant, one or more biologically active substances, one or more active agent particles, and one or more suspending particles, wherein the active agent particles and suspending particles associate together to co-suspend the biologically active substance.

6. The method of claim 1, wherein the medicament is composed of: a pharmaceutically acceptable propellant, one or more biologically active substances, a preparation selected from the group consisting of liposomes and microspheres, and wherein the biologically active substance is first contacted with the liposomes or microspheres in an aqueous medium before being propelled by the propellant.

7. In accordance with the present invention, a method using an exhaler having a body and a nozzle is provided for applying a medicament to the Eustachian tube of a mammal having nostrils for subsequent venous absorption into the mammal's cerebrospinal venous system (CVCS), whereby the exhaler is capable of exerting pressure force and has a medicament reservoir coupled to this pressure force, the exhaler's body is adapted to receive, and block any exhalation through, the mammal's nostrils and the exhaler's nozzle is adapted to receive, and transmit medicaments to, the mammal's Eustachian tube opening, the method comprising: with the body of the exhaler blocking the nostrils and the nozzle of the exhaler adjacent to the opening of the Eustachian tube, performing a Valsalva maneuver to open the Eustachian tube of the mammal and then using the pressure force of the exhaler to transfer the medicament from the reservoir and through the nozzle into the Eustachian tube of the mammal for absorption into the CVCS, which venously drains the Eustachian tube.

8. The method of claim 7, wherein the pressure force of the exhaler that transfers the medicament from the reservoir and through the nozzle into the Eustachian tube of the mammal for absorption into the CVCS has an electromechanical actuating means coupled to an exhalation sensor for sensing the exhalation of the mammal, and the reservoir has a meter for metering an amount of medicament, wherein the actuation of the pressure force used to transmit the metered amount of medicament to the mammal is directly or indirectly responsive to the exhalation sensor and the exhalation sensor actuates the meter at a predetermined trigger point in time relative to the mammal's Valsalva maneuver.

9. The method of claim 7, wherein the medicament is a suspension medium composed of a pharmaceutically acceptable propellant; at least one biologically active substance; at least one active agent particle; and at least one suspending particle, wherein the active agent particle and suspending particle associate together to co-suspend the biologically active substance.

10. The method of claim 7, wherein the medicament is composed of: a pharmaceutically acceptable propellant, one or more biologically active substances, a preparation selected from the group consisting of liposomes and microspheres, wherein the biologically active substance is first contacted with the liposome or microsphere preparation in an aqueous medium before being propelled by the propellant.

11. In accordance with the present invention, an exhaler capable of exerting pressure force, for use in conjunction with a Valsalva maneuver to open the mammal's Eustachian tube, is provided for applying a medicament to the cerebrospinal venous system (CVCS) of a mammal, the exhaler comprising: a body adapted to receive, and block any exhalation through, the mammal's nostrils, a medicament reservoir coupled to the pressure force, and a nozzle adapted to receive, and transmit medicaments to, the mammal's Eustachian tube opening, wherein, with the body of the exhaler blocking the mammal's nostrils and the nozzle of the exhaler is adjacent to the now-opened Eustachian when the Valsalva maneuver is performed to open the Eustachian tube, the pressure force of the exhaler transfers the medicament from the reservoir and through the nozzle to the now open Eustachian tube for absorption into the CVCS, which venously drains the Eustachian tube.

12. The exhaler of claim 11, wherein the exhaler has both a meter, that fluidly communicates selectively between the reservoir and the mammal, for metering an amount of medicament to the pressure force of the exhaler and a electromechanical actuating means coupled to an exhalation sensor for sensing the exhalation of the mammal, wherein the actuation of the pressure force used to transmit the metered amount of medicament from the reservoir to the mammal is responsive to the exhalation sensor and the electromechanical actuating means actuates the meter at a predetermined trigger point in time relative to the mammal's Valsalva maneuver exhalation.

13. The exhaler of claim 11, wherein the pressure force of the exhaler is supplied by the mammal.

14. The exhaler of claim 11, wherein the medicament is a suspension medium composed of a pharmaceutically acceptable propellant; one or more biologically active substances; one or more active agent particles; and one or more suspending particles, wherein the active agent particles and suspending particles associate together to co-suspend the biologically active substance.

15. The exhaler of claim 11, wherein the medicament is composed of: a pharmaceutically acceptable propellant, one or more biologically active substances, a preparation selected from the group consisting of liposomes and microspheres, wherein the biologically active substance is first contacted with the preparation in an aqueous medium before being propelled by the propellant.

16. The exhaler of claim 12, wherein the exhalation sensor is selected from the group consisting of: a exhalation-movable element which is movable in response to the exhalation of the mammal; a pressure sensor for sensing the pressure profile associated with the exhalation of the mammal; a airflow sensor for sensing the airflow profile associated with the exhalation of the mammal; a temperature sensor for sensing the temperature profile associated with the exhalation of the mammal; and, a moisture sensor for sensing the moisture profile associated with the exhalation of the mammal.

17. The exhaler of claim 12, wherein the exhalation-movable element is selected from the group consisting of a vane, a sail, a piston, a diaphragm, a bourbon tube, a bellows and an impeller.

18. The exhaler of claim 12, wherein the electromechanical actuating means is selected from the group consisting of: a spring and/or a lever, a solenoid, a wire, a strip, a coil, and, a tube; and, further, is coupled, and responsive to, a tilt sensor, wherein the actuation of the pressure force used to transmit the metered amount of medicament from the reservoir to the mammal is limited by the tilt sensor to a inclination range of between substantially zero to substantially sixty degrees relative to the sagittal and coronal planes of the mammal.

19. The exhaler of claim 18, wherein the electromechanical actuating means is composed of an alloy selected from the group consisting of: an alloy which is reversibly deformable in response to heat; and, an alloy which is reversibly deformable in response to a magnetic field.

20. The exhaler of claim 16, wherein the pressure sensor for sensing the mammal's exhalation is selected from the group consisting of: a piezoelectric sensor; a piezoresistive strain gauge; a capacitive pressure sensor; an optical sensor; a resonant sensor; a thermal pressure sensor; and, a ionization pressure sensor.
